(19) United States
(12) Patent Application Publication
(10) Pub. No.: US 2013/0289321 A1
(43) Pub. Date: Oct. 31, 2013

(54) METHOD OF CARRYING OUT CC-COUPLING REACTIONS

(71) Applicants: Eric Gaigneaux, Chastre (BE); Marc Jacquemin, Rochefort (BE); Damien Hauwaert, Nivelles (BE)

(72) Inventors: Eric Gaigneaux, Chastre (BE); Marc Jacquemin, Rochefort (BE); Damien Hauwaert, Nivelles (BE)

(73) Assignee: UNIVERSITE CATHOLIQUE DE LOUVAIN, LOUVAIN LA NEUVE (BE)

(21) Appl. No.: 13/996,956

(22) PCT Filed: Dec. 22, 2011
(86) PCT No.: PCT/EP2011/073790

§ 371 Date: Jun. 21, 2013

(30) Foreign Application Priority Data
Dec. 22, 2010 (EP) 10196661.2

(51) Int. Cl. C07C 1/26

(52) U.S. Cl. CPC C07C 1/26
USPC 585/25

(57) ABSTRACT

The present invention is directed to a method of carrying out Suzuki-Miyaura CC-coupling reactions, including reacting an aryl halide with an aryl boronic acid in an organic solvent in the presence of a carbon supported palladium catalyst and a base, wherein the reactions are carried out at constant pH. The invention is also directed to a palladium on carbon catalyst suitable for catalyzing Suzuki-Miyaura CC-coupling reactions.

The present invention is directed to a method of carrying out C—C coupling reactions in the presence of a carbon supported palladium (Pd/C) catalyst and a base.

### BACKGROUND OF THE INVENTION

Chemical reactions aiming to couple carbon atoms are important methodologies for the preparation of organic molecules. These reactions have recently emerged and are often a crucial step in the synthesis of many molecules, especially for the synthesis of pharmaceuticals such as Vancomycine (antibiotic), Steganone, Steganacine (anticancer) or Korupensamine (antimalarial). These reactions are also essential for the synthesis of pesticides (Boscalid) and materials such as liquid crystals, organic conductive materials and semi-conductors. The demand for these molecules is getting more and more important.

Suzuki-Miyaura's reaction was published at first in 1979 by the 2010 Nobel prize winner Akira Suzuki et al. (Miyaura N., Yamada K., Suzuki A., Tetrahedron Lett. 20, 1979, 3437). It creates an aryl-aryl bond in the presence of a palladium catalyst. It allows the combination of an aryl- or vinyl-boronic acid via the boronate group with vinyl or aryl halides (Felpin F.-X., Ayad T. and Mitra S., Eur. J. Org. Chem., 2006, 2679). The reactivity of the aryl halide depends very strongly on the nature of the halides: I>Br>>Cl. The iodides and bromides are usually used, on the other hand the chlorides are less reactive but more and more research is carried out about these molecules (Felpin F.-X., Ayad T. and Mitra S., Eur. J. Org. Chem., 2006, 2679; Simeone J. P., Sowa Jr. J. R., Tetrahedron 63, 2007, 12646-12654).

Suzuki-Miyaura's reaction is carried out in an organic solvent in the presence of a base, which is added at once at the beginning of the reaction and consumed as the reaction goes on.

Suzuki-Miyaura's reaction is one of the most popular reactions for the production of biaryls for several reasons: (i) using mild conditions; (ii) using of boron compounds which are stable, available and have a low toxicity and (iii) a wide range of substrates with various functional groups can be used (Felpin F.-X., Ayad T. and Mitra S., Eur. J. Org. Chem., 2006, 2679; Kotha S., Lahiri K., Kashinath D., Tetrahedron, 58, 2002, 9633).

Suzuki-Miyaura's reaction is traditionally carried out with homogeneous palladium catalysts. These catalysts are soluble complexes of palladium associated to ligands of the arylphosphine type such as the tetrakis(triphenylphosphine)palladium (Pd(PPh₃)₄) (Lu G., Franze R., Zhanga Q., Xua Y., Tetrahedron Lett. 46, 2005, 4255; Amatore, C., Pflüger, F. Organometallics 9, 1990, 2276). Good yields are obtained with these ligands but they often need a big quantity of catalysts (1 to 10 mol %).

The formation of carbon-carbon bonds under homogeneous catalysis has a high potential. Unfortunately, the use of soluble complexes of palladium shows important drawbacks. Firstly, it is very difficult to recover the homogeneous catalysts which are very expensive and it is also difficult to separate them from the reactants and products. Therefore, this type of catalyst cannot be reused. Product contaminations by traces of dissolved catalyst remaining after separation can also arise. This point is particularly important in the synthesis of pharmaceutical molecules for which the residual metal tolerance is very low (less than 5 ppm). Gradually, organic chemistry is turning towards reusable heterogeneous catalysts for the economic and efficient use of raw materials.

Heterogeneous catalysts have been developed to avoid the problems mentioned previously. The main advantages of those catalysts are that they can be recovered by simple filtration or decantation at the end of the reaction and that there is no more product contamination by metals. The catalyst can also be recycled for further reactions. The most common heterogeneous catalyst used for the Suzuki-Miyaura reaction is palladium supported on activated carbons (Pd/C) (Felpin F.-X., Ayad T. and Mitra S., Eur. J. Org. Chem., 2006, 2679; Simeone J. P., Sowa Jr. J. R., Tetrahedron 63, 2007, 12646-12654; Lu G., Franze R., Zhanga Q., Xua Y., Tetrahedron Lett. 46, 2005, 4255). However, the activity of those catalysts is very low.

As a summary, there is still a stringent need for improvements of the Suzuki-Miyaura reaction that combine the advantages of homogenous palladium catalysts and of supported palladium catalysts.

### SUMMARY OF THE INVENTION

After long and intensive research, the present inventors found that the activity of palladium on carbon (Pd/C) catalysts in Suzuki-Miyaura reactions is significantly improved when the reactions are carried out at constant pH.

In a general aspect, the invention thus provides a method of carrying out Suzuki-Miyaura reactions, comprising reacting an aryl halide with an aryl boronic acid, preferably phenyl boronic acid, or an ester thereof in an organic solvent in the presence of a carbon supported palladium catalyst and a base, characterized in that said reactions are carried out at constant pH.

In another aspect, the present invention uses a palladium on carbon catalyst, wherein the palladium is supported on a low specific surface carbon having a specific surface of less than 500 m²/g, preferably less than 200 m²/g, more preferably about 100 m²/g.

### DETAILED DESCRIPTION OF THE INVENTION

As noted above, the invention relates to a method of carrying out Suzuki-Miyaura reactions, comprising reacting an aryl halide with an aryl boronic acid or an ester thereof in an organic solvent in the presence of a carbon supported palladium catalyst and a base, characterized in that reaction is carried out at constant pH.

More specifically, the Suzuki-Miyaura reactions according to the invention mean reacting a compound of general formula A with a compound of general formula B, in an organic solvent in the presence of a carbon supported palladium catalyst and a base, to furnish a compound of general formula C as depicted in scheme 0 below:

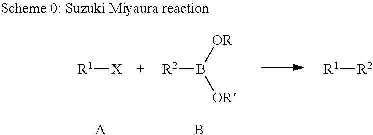

wherein R¹ is aryl;
X is halo;
R² is aryl;
R and R′, identical or different, are independently selected from H, linear or branched C1-C6 alkyl, or R and R′ form together a C2-C5 alkylene chain optionally substituted by one or more C1-C2 alkyl group, thus forming a cyclic boronate group, or R and R′ form together a phenylene ring wherein the oxygen atoms are attached at positions 1 and 2 thereof, thus forming a cyclic pinacol boronate.

The term “halo” according to the invention means fluoro, chloro, bromo, or iodo. Preferred halo groups are bromo and iodo. More preferably, the halo group is bromo.

The term “halide” according to the invention means fluoride, chloride, bromide, or iodide. Preferred halide groups are bromide and iodide. More preferably, the halide is bromide.

The term “aryl” as used herein by itself or as part of another group refers to a substituted or unsubstituted polyunsaturated, aromatic hydrocarbyl group having a single ring (i.e. phenyl) or multiple aromatic rings fused together (e.g. naphtyl) or linked covalently, typically containing 5 to 12 atoms; preferably 6 to 10, wherein at least one ring is aromatic. The aromatic ring may optionally include one to two additional rings (either cycloalkyl, heterocyclyl or heteroaryl) fused thereto. Aryl is also intended to include the partially hydrogenated derivatives of the carbocyclic systems enumerated herein. Non-limiting examples of aryl comprise phenyl, tolyl, biphenylyl, biphenylenyl, 5- or 6-tetralinyl, naphthalen-1- or -2-yl, 4-, 5-, 6 or 7-indenyl, 1-2-, 3-, 4- or 5-acenaphtylenyl, 3-, 4- or 5-acenaphtenyl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3-, 4- or 5-pyrenyl. Preferred aryl groups are phenyl, tolyl, and naphtyl, more preferably phenyl and tolyl.

As indicated above, the aryl moieties can be substituted by one or more substituent(s). Non limiting examples of aryl and heteroaryl substituents are cyano, nitro, substituted and unsubstituted linear or branched C1-C6 alkyl, substituted and unsubstituted linear or branched C1-C6 alkoxy, hydroxyl, carboxaldehyde, carboxy, amino, amides, sulfonamides, ureas, carbamates, and derivatives thereof. Preferred substituents are C1-C2 alkyl, preferably methyl.

The expression “aryl halide” refers to an aryl radical having the meaning as defined above wherein one or more hydrogens are replaced with a halo as defined above. Non-limiting examples of such aryl halides include fluorobenzene, chlorobenzene, bromobenzene, iodobenzene, 2-bromotoluene, 3-bromotoluene, 4-bromotoluene, 2-iodotoluene, 3-iodotoluene, 4-iodotoluene.

The term “alkyl” by itself or as part of another substituent refers to a hydrocarbyl radical of Formula CₙH₂ₙ₊₁ wherein n is a number greater than or equal to 1. Generally, alkyl groups of this invention comprise from 1 to 6 carbon atoms, preferably from 1 to 4 carbon atoms, more preferably from 1 to 3 carbon atoms, still more preferably 1 to 2 carbon atoms. Alkyl groups may be linear or branched and may be substituted as indicated herein. Cx-Cy-alkyl refers to alkyl groups which comprise from x to y carbon atoms.

When the suffix “ene” (“alkylene”) is used in conjunction with an alkyl group, this is intended to mean the alkyl group as defined herein having two single bonds as points of attachment to other groups. The term “alkylene” includes ethylene, propylene, and pentylene.

The term “boronic acid” refers to a compound bearing a

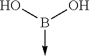

group, wherein the arrow designates the attachment point.

The expression “aryl boronic acid” alone or in combination refers to an aryl radical having the meaning as defined above wherein one or more hydrogens are replaced with a boronic acid as defined above. Non-limiting examples of such aryl boronic acids include phenyl boronic acid.

The expression “ester of boronic acid” refers to a compound bearing a

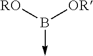

group, wherein the arrow designates the attachment point, and wherein R and R′, are defined as in Formula B above.

Suitable esters of boronic acids are di-(linear or branched C1-C4 alkyloxy)borane derivatives, for instance dimethoxyborane or diethoxyboranes, and boronate esters of 1,3-propanediol, 1,1,2,2-tetramethylethan-1,2-diol (pinacol), 2-methyl-2,4-pentanediol and catechol (2-hydroxyphenol), the boronate ester of pinacol being the preferred ester of boronic acid.

Advantageously, the constant pH is from 8 to 12, preferably from 10 to 11, more preferably about 10.6.

Traditionally, Suzuki-Miyaura reactions are carried out at decreasing pH as the base is added at once at the beginning of the reaction and then consumed as the reaction goes on. Working at constant pH under heterogenous catalysis improves the conversion rate and the selectivity of the Suzuki-Miyaura reaction compared to working at decreasing pH described in literature. The selectivity is defined as the ratio between the number of moles of 4-methylbiphenyl produced and the number of moles of 4-bromotoluene converted. The invention thus provides a method of carrying out Suzuki-Miyaura reactions that combines the advantages of heterogeous catalysis with those of homogenous catalysis: easy recovery (e.g. by filtration or decantation) and recycling of the catalyst due to the use of a supported catalyst and increased activity of the catalysts leading to higher selectivity and conversion rates compared to traditional heterogenous catalysis.

In order to maintain a constant pH, the base is not, as usual, added at once at the beginning of the reaction, but continuously. The base is preferably an inorganic base. Suitable inorganic bases include, but are not limited to K₂CO₃, Na₂CO₃, Cs₂CO₃, NaOH, KOH, NaHCO₃, Na₃PO₄, and KF. In a preferred embodiment, the base is selected from the group consisting of K₂CO₃, NaOH, KOH, Na₂CO₃, and NaHCO₃.

A wide variety of organic solvents can be used for Suzuki-Miyaura reactions. Suitable organic solvents include, but are not limited to, DMF (dimethylformamide), DME (dimethoxyethane), DMA (dimethylacetamide), NMP (N-methylpyrrolidone), THF (tetrahydrofuran), toluene, methanol, ethanol, iso-propanol, n-butanol, water, and mixtures thereof. In a preferred embodiment, the solvent is DMF, alone or in a mixture with water. More preferably, the solvent is a mixture of DMF and water.

The carbon support is either a high specific surface carbon or a low specific surface carbon. Within the meaning of the present invention, high specific surface carbons are those having a specific surface of more than 500 m²/g, preferably more than 1000, and low specific surface carbons are those having a specific surface of less than 500 m²/g, preferably less than 200 m²/g, more preferably about 100 m²/g.

In one embodiment, the carbon support is a high specific surface carbon as defined above, preferably activated carbon.

In another embodiment, the carbon support is a low specific surface carbon as defined above, preferably carbon black. Carbon black [C.A.S. NO. 1333-86-4] is virtually pure elemental carbon in the form of colloidal particles that are produced by incomplete combustion or thermal decomposition of gaseous or liquid hydrocarbons under controlled conditions. Carbon black is a form of amorphous carbon that has a low specific surface compared to that of activated carbon.

The catalysts used in the method of the invention can be prepared according to the methods known in the art, such as wet impregnation.

As already mentioned above, the invention also pertains to a carbon supported palladium catalyst, wherein the palladium is supported on a low specific surface carbon having a specific surface of less than 500 m²/g, preferably less than 200 m²/g, more preferably 80 to 100 m²/g. In a preferred embodiment, the low specific surface carbon is carbon black. The main advantage of carbon black is its low specific surface and thus its larger pores than activated carbon. The larger pores reduce or even avoid problems of diffusion.

The present invention will be better understood with reference to the following examples. These examples are intended to be representative of specific embodiments of the invention, and are not intended as limiting the scope of the invention.

### EXAMPLES

### Example 1

### Preparation of the Catalysts

Two types of carbon were used as catalyst support:
carbon LSS “Low Specific Surface” (carbon black: Printex U, Degussa, 970618214) having a specific surface area about 91 m²/g and
carbon HSS “High Specific Surface” (activated carbon: Merck, 102518) having a specific surface area about 1263 m²/g.

Before the impregnation, the supports were dried in air for 15 h at 110° C.

0.3334 g of palladium chloride (PdCl₂, Aldrich, 205885) was dissolved in an aqueous solution of HCl 0.1M. 4 g of dried support was mixed with the impregnation solution for 10 min under magnetic stirring. Then, water was removed under reduced pressure in a rotavapor at 40° C. The recovered solid was dried at 110° C. overnight. The catalysts were synthesized with different loadings of Pd in mass comparing to the support: 1, 3, 5 and 10% for the LSS carbon and a loading of 5% for the HSS carbon. Catalysts are denoted Pd(x %)/C−y, where “x” represents the Pd weight loading in % and “y” is the support, LSS or HSS.

### Example 2

### Characterization of the Catalysts

The chemical composition of catalysts was measured by Inductively Coupled Plasma Atomic Emission Spectroscopy (ICP-AES) on an Iris Advantage apparatus from Jarrel Ash Corporation. The catalyst was dried at 110° C. prior to measurement.

Textural analysis of the catalyst was carried out on a Micromeritics Tristar 3000 equipment using N₂ adsorption/desorption at liquid N₂ temperature, working with relative P/P₀ pressures in the range of 10⁻² to 1.0. Before the measurements, 150 mg of the samples were degassed at 150° C. overnight under a vacuum (50 mTorr (0.067 mBar)). The specific surface area was calculated from the amount of N₂ adsorbed by using 5 points with relative P/P₀ pressures between 5*10⁻² and 0.3 (BET theory). BJH equations were used to determine the distribution of pores diameter and the total pore volume was assessed from the amount of nitrogen adsorbed at P/P₀=0.98.

X-ray diffraction (XRD) analysis were performed on the fresh catalyst on a Siemens D5000 diffractometer using the K_{α} radiation of Cu (λ=1.5418 Å). The 2θ range was scanned between 5 and 90° at a rate of 0.02°·s⁻¹. Identification of the crystalline phases was carried out using the ICDD-JCPDS database.

Surface characterization of the fresh catalyst was done by X-ray photoelectron spectroscopy (XPS) measurements on a Kratos Axis Ultra spectrometer (Kratos Analytical—Manchester—UK) equipped with a monochromatized aluminium X-ray source (powered at 10 mA and 15 KV). The pressure in the analysis chamber was around 10⁻⁶ Pa. The analyzed area was 700 μm×300 μm. The pass energy of the hemispherical analyser was set at 40 eV. Charge stabilisation was achieved by using the Kratos Axis device. The electron source was operated at a filament current of 1.8 A and a bias of −1.1 eV. The charge balance plate was set at 2.8 eV. The sample powders were pressed into small stainless steel troughs mounted on a multi specimen holder. The following sequence of spectra was recorded: survey spectrum, C 1s, O 1s, Al 2p and Pd 3d and C 1s again to check the stability of charge compensation in function of time and the absence of degradation of the sample during the analyses. The binding energy (BE) values were referred to the C—(C,H) component of the C 1s peak fixed at 284.8 eV. The spectra were decomposed with the CasaXPS program (Casa Software Ltd., UK) with a Gaussian/Lorentzian (70/30) product function. Molar fraction were calculated using peak areas normalised on the basis of acquisition parameters, sensitivity factors and transmission factors provided by the manufacturer.

Dispersion of Pd was determined by using carbon monoxide chemisorption. The CO chemisorption measurements were conducted at 35° C. using a Micromeritics Pulses Chemisorb 2700 apparatus equipped with a TCD detector. The sample (150 mg) was reduced at 400° C. under a pure flow of Hydrogen (Praxair, 99.999%) for 2 hours and then flushed for 1 hour under He and finally cooled down to 35° C. Several injections of a known volume of CO (185 μl) are sent on the samples. The apparatus gives a peak area which corresponds to the non-adsorbed CO. When there is no more adsorption of CO, the value of the peak area corresponds to the volume of the injection loop. By simple subtraction, the volume of CO which was adsorbed at each injection on palladium was calculated. Thereafter, those values were added for all injections of CO to obtain the total volume of CO adsorbed. This total is corrected so as to adjust the standard conditions of pressure and temperature. The stoechiometry of adsorption is equaled to one adsorbed CO molecule per atom of active metal (Pd).

The quantity of CO chemisorbed is related to the dispersion (D) with the following equation:

$$D = \frac{\sum Δ S \cdot {V}_{CO} \cdot MM \cdot A}{{S}_{max} \cdot B \cdot m}$$

With:
ΣΔS: the sum of the difference between the values of area measured before the saturation of the sample and the value of the maximal area which corresponds to the saturation,
V_{CO}: the volume injected CO (1),
A: a conversion factor (273/(298×22.4)) (mol·l⁻¹),
MM: molar mass of the metal (106.42 g·mol⁻¹),
Sₘₐₓ: area of the peak which corresponds to the volume of CO injected in one injection,
B: loading of the metal deposed on the support (%),
m: mass of the analyzed sample (g).

The particle size of the Pd on the catalysts is estimated by the following equation:

$$d ( m ) = \frac{500 \times MM}{ρ \times σ \times D}$$

d: Particle size (m)
MM: Molar mass of the metal (Pd: 106.42 g·mol⁻¹);
ρ: Density of the metal (Pd: 12×10⁶ g·m⁻³);
σ: Surface of one mole of the metal (Pd: 47800 m²ₘₑₜₐₗ·mol⁻¹);
D: Dispersion (%).

The results of chemical analyses performed on catalysts supported on carbons are presented in Table 1. Dispersions (%) and the Pd particles size (nm) are also presented.

TABLE 1
Chemical composition of catalysts supported on carbons

| | Pd (wt. %) | Dispersion | Particles size |
|---|---|---|---|
| Catalysts | experimental | (%) | (nm) |
| Pd(1%)/C-LSS | 0.8 | 6 | 15 |
| Pd(3%)/C-LSS | 2.7 | 7 | 13 |
| Pd(5%)/C-LSS | 5.4 | 7 | 13 |
| Pd(10%)/C-LSS | 8.3 | 7 | 13 |
| Pd(5%)/C-HSS | 4.4 | 6 | 15 |

Table 2 shows that the specific surface area (SSA) remains identical for the LSS support and for the catalysts prepared therewith, except for the Pd(10%)/C−LSS which shows a decrease. Unlike, the SSA decreases significantly for Pd(5%)/C−HSS as compared to the fresh support. The SSA developed by the micropores represents the major part of the total specific surface area.

TABLE 2
Textural analysis (BET) of the carbon supports and catalysts

| | Specific | Porous | | Microporous |
|---|---|---|---|---|
| | surface area | volume | Pores size | specific surface |
| Catalysts | (m²/g) | (cm³/g) | (Å) | area (m²/g) |
| C-LSS | 91 | 0.80 | 376 | 19 |
| Pd(1%)/C-LSS | 87 | 0.77 | 353 | 18 |
| Pd(3%)/C-LSS | 99 | 0.71 | 257 | 12 |
| Pd(5%)/C-LSS | 99 | 0.84 | 257 | 18 |
| Pd(10%)/C-LSS | 68 | 0.51 | 332 | 9 |
| C-HSS | 1263 | 0.51 | 22 | 718 |
| Pd(5%)/C-HSS | 1088 | 0.48 | 23 | 634 |

In XRD analysis, all samples were characterized by a broad band around 20-25° typical of amorphous solids. No trace of crystalline palladium was found. This fact does not suggest that the Pd is not in crystalline form but it is possible that crystalline domains are present but undetected because they are too small.

The XPS analysis (Table 3) can also estimate the accessibility of Pd on the catalysts by the exploitation of atomic ratios Pd/C and its oxidation state by the exploitation of the Pd 3d peak position. In each analysis, the palladium is in the oxidized form (Pd²⁺). The presence of Cl in some samples can be explained by the fact that the Pd precursor used is PdCl₂ and carbon-based catalysts were simply dried and not calcined, so chlorine could not be volatilized. The amount of Cl is related to the ratio Pd/C, which is quite logical because when we add more Pd, we consequently add more chlorine.

TABLE 3
Analysis of the carbon-based catalysts surface by XPS

| Catalysts | O/C | Pd/C | Cl/C |
|---|---|---|---|
| C-LSS | 0.08 | 0.00 | 0.00 |
| Pd(1%)/C-LSS | 0.10 | 0.04 | 0.01 |
| Pd(3%)/C-LSS | 0.12 | 0.10 | 0.01 |
| Pd(5%)/C-LSS | 0.17 | 0.22 | 0.03 |
| Pd(10%)/C-LSS | 0.26 | 0.43 | 0.06 |
| C-HSS | 0.05 | 0.00 | 0.00 |
| Pd(5%)/C-HSS | 0.16 | 0.14 | 0.02 |

### Example 3

### Catalytic Tests

### Suzuki-Miyaura's Catalytic Tests Reactions

The five Pd/C catalysts prepared in Example 1 were tested in the following Suzuki-Miyaura test reaction. The reactants were 4-bromotoluene and phenylboronic acid. The desired product is 4-methylbiphenyl.

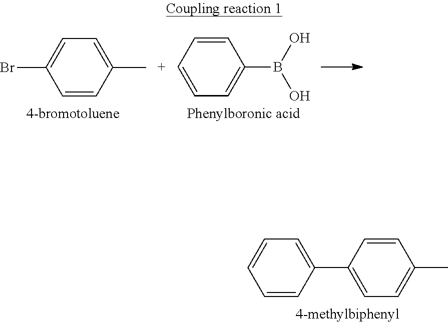

A 5 neck round flask was placed in an oil bath. To reduce the loss of reagents by evaporation, a condenser is connected to the reactor. The reaction temperature was measured using a thermometer in contact with the reaction medium.

All catalysts were sieved and selected in the 100-200 μm granulometric fractions. The catalytic tests were carried out in the 5 neck flask with mechanical agitation (210 rpm). The solid reagents, 1.5000 g of 4-bromotoluene and 1.3256 g of phenylboronic acid, 0.1750 g of catalyst and 1.0810 g of biphenyl, the internal standard, were introduced first, followed by 60 ml of dimethylformamide (DMF). The flask was placed in a thermostatic oil bath and the reaction was performed under a nitrogen atmosphere. All catalytic tests have been realized by using a basic solution of K₂CO₃ (5 g of K₂CO₃/100 ml of water buffered with HCl at 10.6) and a reaction temperature of 95° C.

Once the working temperature (95° C.) was reached, the desired amount (15 ml) of K₂CO₃ solution was added manually using a syringe or with an automatic titrator. The pH was monitored and the base added during the catalytic test so as to maintain the pH at 10.6.

The reaction begins when the K₂CO₃ solution was added. Samples (0.5 ml) were taken every 1 min 30 after adding the base until 10 min 30 and after which a last aliquot was taken at 15 min. Before analysis, samples are filtered (PTFE syringe filters, filtration threshold 0.45 μm) to remove the catalyst.

For quantification, an internal standard (biphenyl) was used and an appropriate calibration has been performed. Biphenyl was chosen as internal standard because it has a chemical 20 structure very close to the desired reaction product, namely 4-methylbiphenyl. Tests were conducted to check that the internal standard did not react with any compounds present in the reaction, namely 4-bromotoluene, phenylboronic acid, 4-methylbiphenyl, the base and DMF. For the analysis of samples, the sample was diluted (1:9 vol) in dichloromethane (JANSSEN CHIMICA, 1134696) and analyzed by a CP-3800 gas chromatography (GC) apparatus from Varian equipped with an autosampler (CP-8200 Varian) on a column CP-Sil 5CB (50 m×0.32 mm×0.4 μm) Varian. The initial pressure is 15 psi (1.034 bar) in the column. The temperature program applied for the separation of reactants and products in the chromatography column is as follows: initial temperature 80° C. is maintained for 3 min, and then a ramp of 30° C./min was applied up to 180° C. This temperature is maintained for 3 minutes. A ramp of 15° C./min is applied to 250° C. for 3 min. Finally, the temperature increases to 300° C. with a ramp of 30° C./min. This temperature is maintained during 3 min. The total duration of the program is 15.76 minutes. The conversion is defined as the ratio “moles of 4-bromotoluene converted/mole of 4-bromotoluene initial*100%”. The selectivity for the 4-methylbiphenyl is defined as the ratio “moles of 4-methylbiphenyl produced/mole of 4-bromotoluene consumed*100%”.

### Test Results

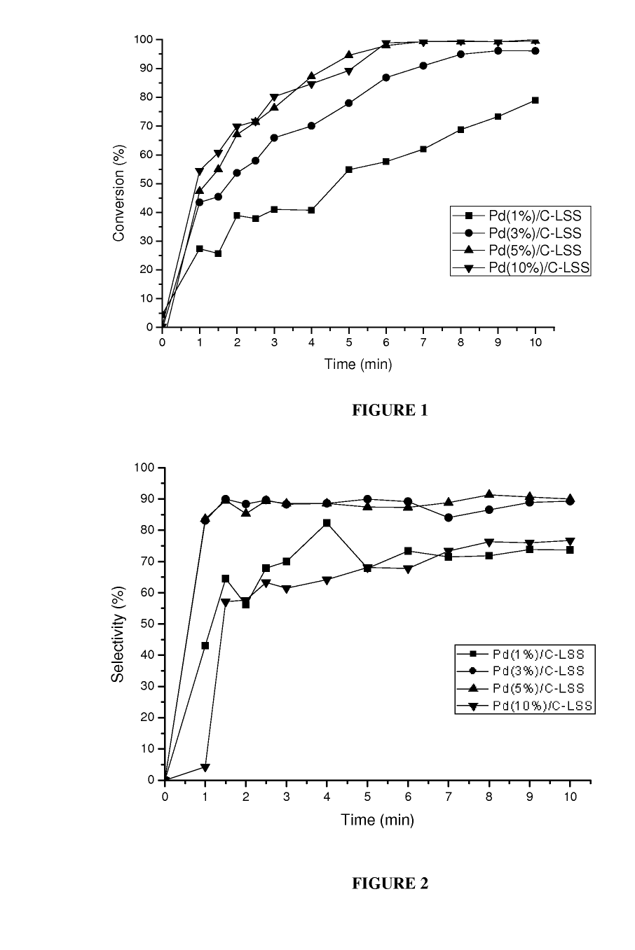

The catalytic performances of the catalysts supported on the LSS carbon with different loadings (1, 3, 5, 10%) have been measured. FIG. 1 and FIG. 2 show respectively the conversion of the 4-bromotoluene and the selectivity to 4-methylbiphenyl depending on the time for those catalysts. The results obtained are also much better than those found in the literature (Felpin F.-X., Ayad T. and Mitra S., Eur. J. Org. Chem., 2006, 2679; Simeone J. P., Sowa Jr. J. R., Tetrahedron 63, 2007, 12646-12654).

Regarding the comparison of a Pd/C−LSS and a Pd/C−HSS catalyst, the conversion of 4-bromotoluene reaches 100% after the same time (6 min), but the activity of the Pd(5%)/C−LSS was higher at the beginning. On the contrary, the production of 4-methylbiphenyl was slightly higher for the Pd(5%)/C−HSS after 6 min of reaction. However after 1.5 min of reaction, the selectivity was the same.

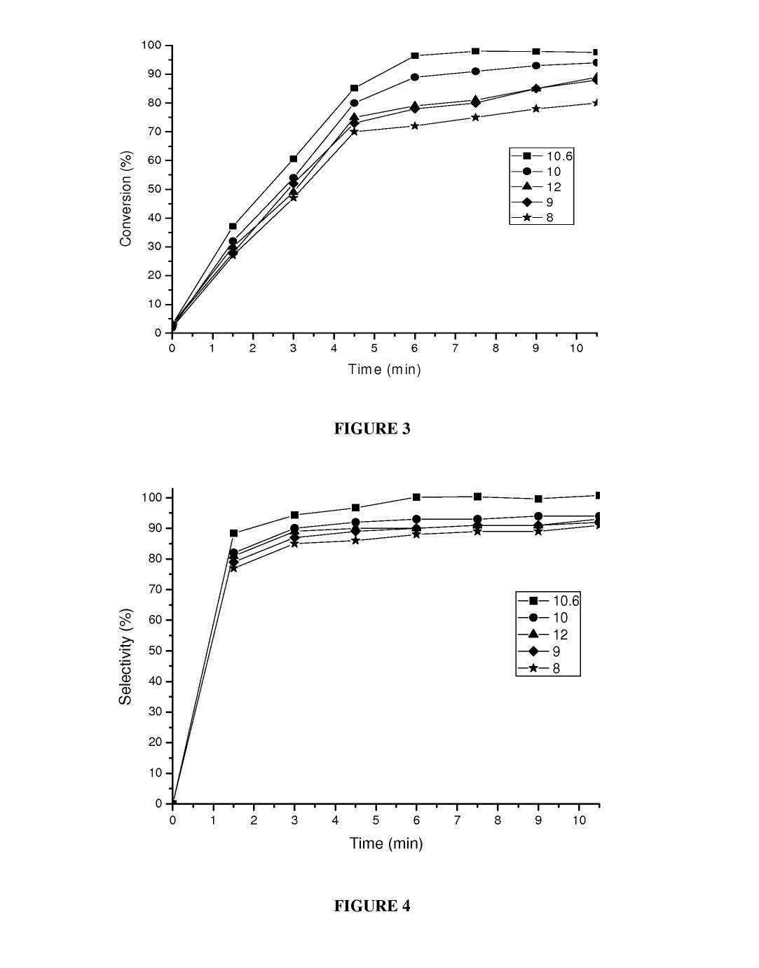

It has been experimentally determined that using a constant pH of 10.6 was the optimum way to introduce the base during the reaction with the carbon-supported catalysts (FIGS. 3 and 4). This is accomplished through the use of an automatic titrator which maintains a constant pH by adding base to replace the one consumed during the reaction.

### Comparative Example 1

The Suzuki-Miyaura test reaction described in Example 3 was carried out using the Pd(5%)/C−LSS catalyst of example 1. In contrast to Example 3, 15 ml of K₂CO₃ solution (5 g/100 ml of water buffered with HCl at 10.6) were added at once at the beginning of the reaction.

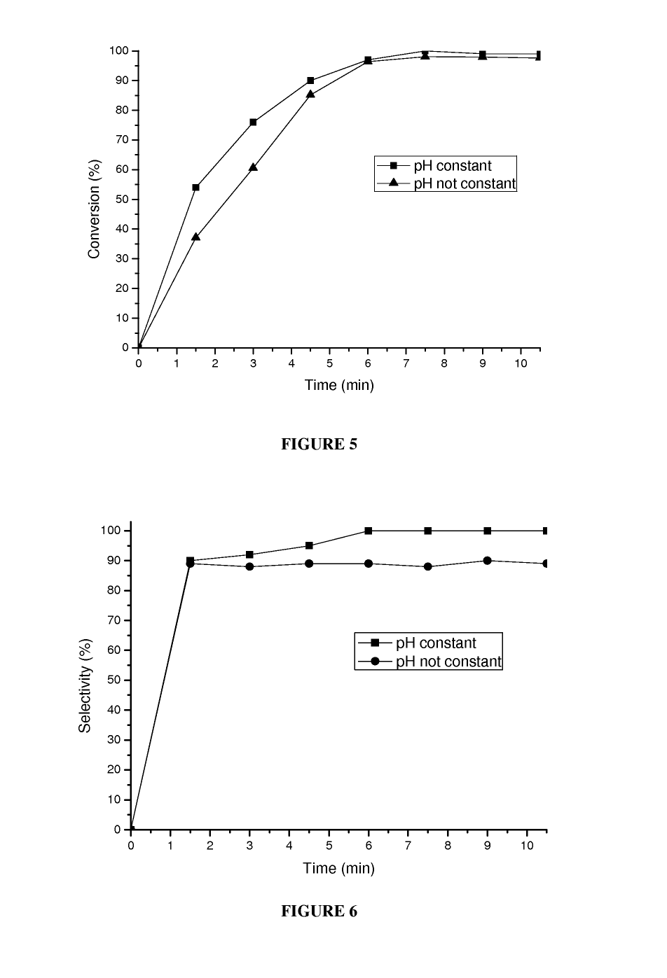

The starting pH was close to 14 and decreased during the reaction. The conversions of both tests (constant pH versus a “classical test” in which the base was added at the beginning of the test and the pH decreased during the test) were identical after 6 min of reaction (FIG. 5). However, the selectivity to 4-methylbiphenyl reached 100% after 6 min when the pH was kept constant at 10.6 (FIG. 6), whereas the selectivity in the “classical test” below 90% after 6 min.

Hence, working at constant pH improves the selectivity of the test reaction while maintaining equivalent conversion rates.

### Example 4

### Catalytic Tests

### Suzuki-Miyaura's Catalytic Tests Reactions

The five Pd/C catalysts prepared in Example 1 were tested in the following Suzuki-Miyaura test reaction (coupling reaction 2). The reactants were 4-bromotoluene and phenylboronic acid pinacol ester. The desired product is 4-methylbiphenyl.

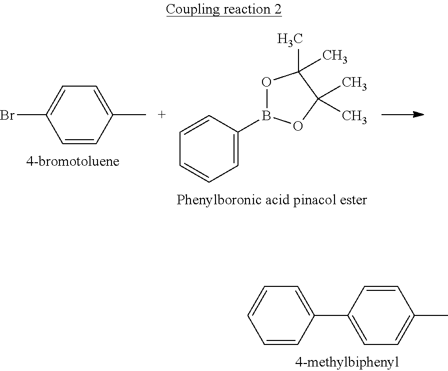

The procedure used in this catalytic test is identical to the one described in Example 3.

### Test Results

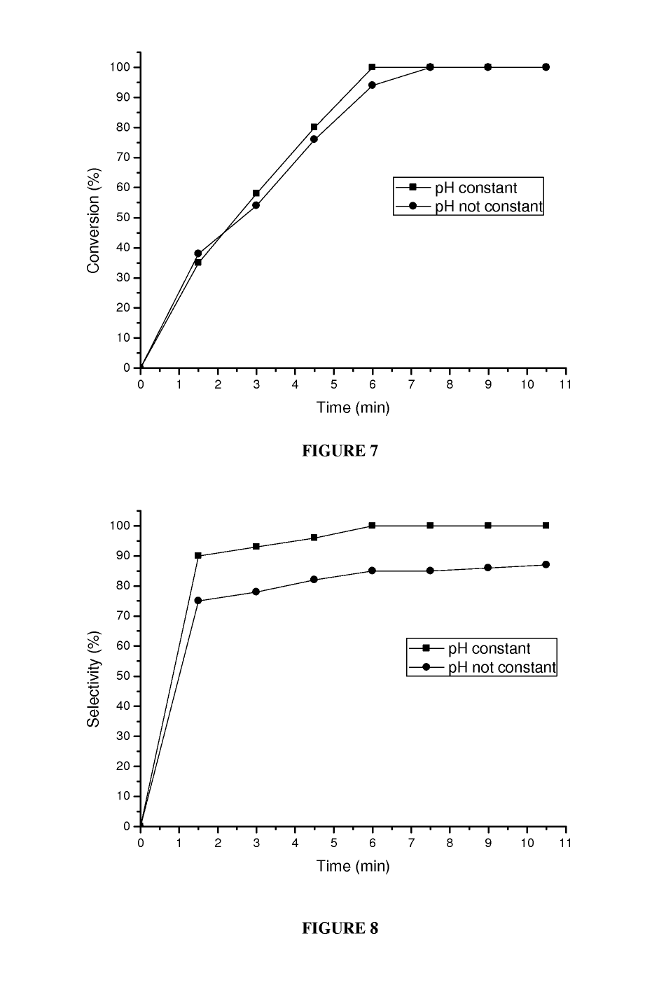

FIGS. 7 and 8 show respectively the selectivity and the conversion obtained for the Pd(5%)/C−LSS catalyst of Example 1 when the pH was kept constant at 10.6.

### COMPARATIVE EXAMPLE 2

The Suzuki-Miyaura test reaction described in Example 4 was carried out using the Pd(5%)/C−LSS catalyst of example 1. In contrast to Example 4, 15 ml of K₂CO₃ solution (5 g/100 ml of water buffered with HCl at 10.6) were added at once at the beginning of the reaction.

The conversions of both tests (constant pH (Example 3) versus a “classical test” in which the base was added at the beginning of the test and the pH decreased during the test (Comparative Example 2)) were identical after 6 min of reaction (FIG. 7). However, the selectivity to 4-methylbiphenyl reached 100% after 6 min when the pH was kept constant at 10.6 (FIG. 8), whereas the selectivity in the “classical test” remained below 90% after 6 min (FIG. 8).

1. Method of carrying out Suzuki-Miyaura CC-coupling reactions, comprising reacting an aryl halide with an aryl boronic acid or an ester thereof in an organic solvent in the presence of a carbon supported palladium catalyst and a base, characterized in that said reactions are carried out at constant pH.

2. The method of claim 1, wherein the constant pH is from 8 to 12.

3. The method of claim 1, wherein the base is an inorganic base.

4. The method of claim 1, wherein the base is selected from the group consisting of K₂CO₃, NaOH, KOH, Na₂CO₃, and NaHCO₃.

5. The method of claim 1, wherein the carbon support of the catalyst is selected from high specific surface carbons having a specific surface of more than 1000 m²/g or low specific surface carbons having a specific surface of less than 500 m²/g.

6. The method of claim 5, wherein the carbon support is selected from high specific surface carbons.

7. The method of claim 5, wherein the carbon support is selected from low specific surface carbons.

8. The method of claim 1, wherein the ester of an aryl boronic acid is the boronate ester of 1,1,2,2-tetramethylethan-1,2-diol (pinacol).

9. The method of claim 1, wherein the aryl halide is reacted with an aryl boronic acid.

10. The method of claim 9, wherein the aryl boronic acid is phenyl boronic acid.

11. The method of claim 1, wherein the pH is from 10 to 11.

12. The method of claim 1, wherein the pH is about 10.6.

13. The method of claim 1, wherein the carbon support is selected from the group consisting of low specific surface carbons having a specific surface of less than 200 m²/g.

14. The method of claim 1, wherein the carbon support is selected from the group consisting of low specific surface carbons having a specific surface of less than 100 m²/g.

15. The method of claim 5, wherein the carbon support is activated carbon.

16. The method of claim 5, wherein the carbon support is carbon black.
